(19) United States
(12) Patent Application Publication
(10) Pub. No.: US 2020/0288676 A1
(43) Pub. Date: Sep. 17, 2020

(54) Method for Pet-Health Awareness with Real-Time Health Monitoring and Illness Notification Functions

(71) Applicant: Farzaneh Pak Parvar, National City, CA (US)

(72) Inventor: Farzaneh Pak Parvar, National City, CA (US)

(21) Appl. No.: 16/353,686

(22) Filed: Mar. 14, 2019

(51) Int. Cl. A01K 29/00; A01K 27/00; A61B 5/00; A61B 5/0205

(52) U.S. Cl. CPC A01K 29/005; A01K 27/001; A61B 5/0082; A61B 5/0077; A61B 5/0205; A61B 5/742; A61B 5/6802

(57) ABSTRACT

The method for pet-health awareness with real-time health monitoring and illness notification functions is implemented with a controller, a wireless communication module, at least one biochemical sensor, a plurality of biometric threshold ranges that is stored within the controller, and a mobile computing device. The biochemical sensor reads a plurality of biochemical signatures which measures the overall health of an animal. The plurality of biochemical signatures is then relayed to a remote server for storage and processed through the controller. If an arbitrary biochemical signature is not within the corresponding threshold range, the controller generates a warning message. The remote server then relays the warning message to the mobile computing device from the wireless communication module.

### FIELD OF THE INVENTION

The present invention generally relates to a method of promoting pet-health awareness by monitoring the vital signs of the pet. More specifically, the present invention utilizes a plurality of biochemical sensors to measure the pet's vital signs and notify the pet's owner of any abnormality or illness.

### BACKGROUND OF THE INVENTION

The field of healthcare for pet animals is lacking and under-developed. This is especially alarming as millions of American households keep a dog or a cat as a pet. In fact, there are over 89 million dogs in United States alone. Most pets are also treated as part of the family and often share the same living space as the other members of the family. However, the close proximity between pets and the family can also cause illnesses to spread from the pet to members of the family. This is especially dangerous to children who are frequently in contact with the pets and have weak immune systems.

However, many pet-owners are incapable of detecting when their pets are sick. As a result, most pet-owners are unaware of the overall health of their pets. Additionally, many pet-owners fail to take their pets to their veterinarians for frequent checkup which leads to untreated illnesses. As a result, the chance of their pets having an illness is very high which jeopardizes the health of their whole family. Thus, it is vital for pet-owners to keep their pets healthy so as not to spread illnesses from their pet to their families and themselves

To remedy this, it is important for pet-owners to be always aware of their pet's health. This facilitates early detection and treatment of pet ailments which can often be more affordable for the pet-owner and safer for the pet. One of the most reliable way of measuring the overall health of an animal is by measuring the body temperature and the heartbeat. A high body temperature and a high heartbeat can often indicate life-threatening illnesses, which may require immediate attention.

As such, the present invention is a method for pet-health awareness with real-time monitoring and illness notification functions. The present invention utilizes a biochemical sensor mounted on the collar worn around the pet's neck. The biochemical sensor contacts the pet's skin and measures the heartrate and the body temperature of the pet. The readings are sent to a remote server which stores and processes the data. If the heartrate or the body temperature is abnormally high or low, the remote server sends a warning to the pet-owner. This prompts the pet-owner to take the pet for a checkup, thereby preventing the illness from getting any worse.

### BRIEF DESCRIPTION OF THE DRAWINGS

FIG. 1 is a schematic diagram illustrating the wireless communication module relaying information to the mobile computing device through the remote server.

FIG. 2 is a flowchart illustrating the overall process of the present invention.

FIG. 3 is a deployment diagram showing the flow of information from the health monitoring device to the mobile app.

FIG. 4 is a flowchart illustrating the sub-processes for providing the plurality of biometric threshold ranges stored within the controller.

FIG. 5 is an activity diagram showing the process for logging into the user account.

FIG. 6 is a flowchart illustrating the sub-processes for providing the plurality of biometric threshold ranges via a user account managed by the remote server.

FIG. 7 is a flowchart illustrating the sub-processes for generating the analytics report based on the plurality of biochemical signatures.

FIG. 8 is a flowchart illustrating the sub-processes for generating the warning message in accordance to the heartrate reading.

FIG. 9 is a flowchart illustrating the sub-processes for generating the warning message in accordance to the body temperature reading.

FIG. 10 is a flowchart illustrating the sub-processes for providing a diagnosis by comparing the plurality of biochemical signatures to the plurality of illness profiles.

### DETAILED DESCRIPTION OF THE INVENTION

All illustrations of the drawings are for the purpose of describing selected versions of the present invention and are not intended to limit the scope of the present invention.

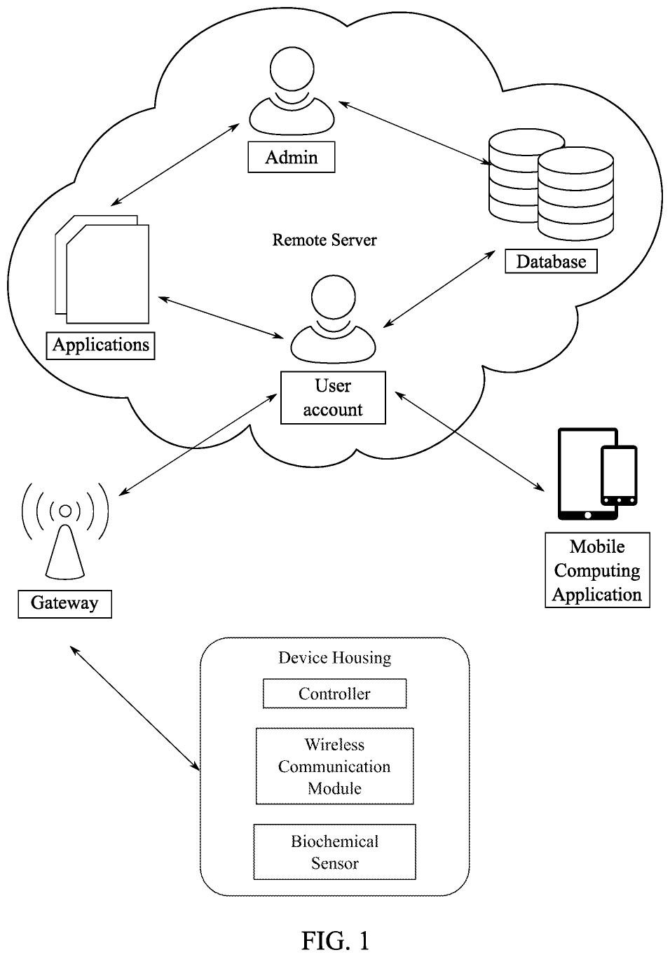

Referring to FIG. 1, the present invention is a computer-executable method for pet-health awareness with real-time health monitoring and illness notification functions. The method is stored in a non-transitory computer readable medium and executed by one or more processors included as part of one or more independent computing systems. The preferred modality of the present invention is represented as a computer application executed by one or more mobile computing systems and at least one remote server.

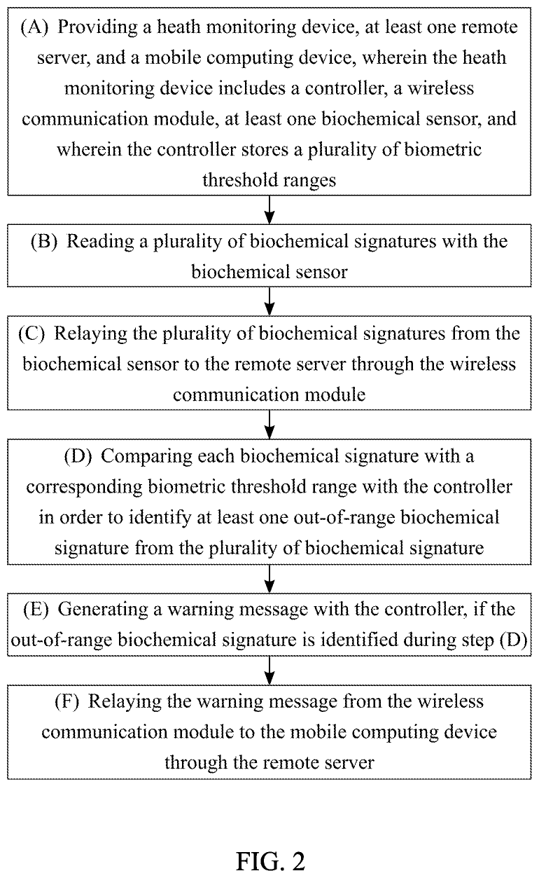

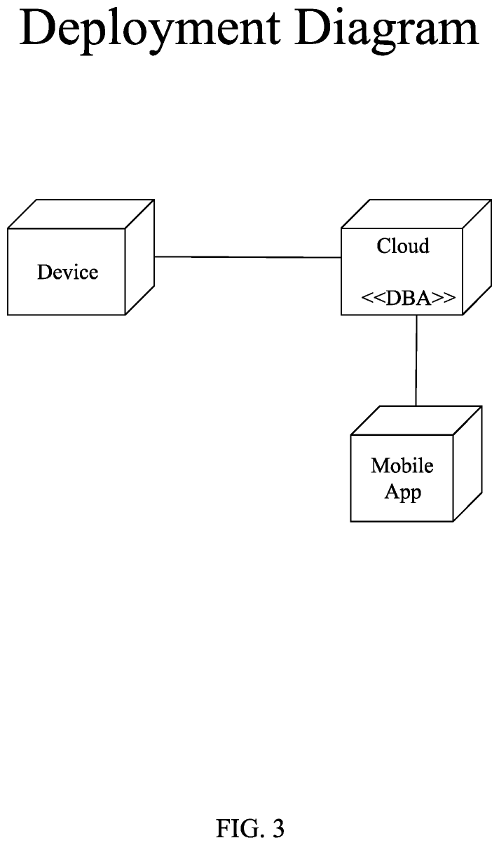

Referring to FIG. 2 and FIG. 3, a health monitoring device, at least one remote server, and a mobile computing device are provided, wherein the health monitoring device includes a controller, a wireless communication module, at least one biochemical sensor, and wherein the controller stores a plurality of biometric threshold ranges (Step A). The controller, the wireless communication module, and the at least one biochemical sensor are mounted within the health monitoring device that protects the enclosed components from environmental elements. In one possible embodiment, the health monitoring device is mounted to a pet-collar worn by the pet, thus fastening the health monitoring device to the animal's body. This allows the biochemical sensor to take readings of the animal's body. The preferred biochemical sensor measures the vital signs of the animal's body to determine the overall health of the animal. Accordingly, the biochemical sensor is mounted externally on the health monitoring device. This positions the biochemical sensor against the skin of the animal, thereby allowing the biochemical sensor to take clear readings.

In one possible embodiment, the biochemical sensor is a touch sensor positioned to contact the skin of an animal kept as a pet. The touch sensor may take biochemical readings that are used to determine the overall health of the animal. The controller is a programmable integrated circuit (IC) that records and processes the readings from the biochemical sensor. Similarly, the wireless communication module connects to the remote server through a wireless local area network (WLAN) or a cellular network. Accordingly, the biochemical sensor reads a plurality of biochemical signatures (Step B). In one possible embodiment, the plurality of biochemical signatures includes, but is not limited to, a heart-beat reading and a body temperature reading. Alternately, the plurality of biochemical signatures may include a blood reading, an electrocardiogram (ECG) reading, a blood pressure reading, and the like.

Subsequently, the wireless communication module relays the plurality of biochemical signatures from the biochemical sensor to the remote server (Step C). In the preferred embodiment, the plurality of biochemical signatures is stored in the remote server. The at least one remote server may be a monolithic or distributed computing system responsible for storing, processing, and outputting data. Alternately, the at least one remote server may be part of a plurality of servers networked together in a cloud computing platform. This may allow the plurality of servers to share databases, applications, and computing resources with each other to optimize performance and response times. Users have the ability to access historical records of the plurality of biochemical signatures through client computing devices. In the preferred embodiment, a prospective user uses the mobile computing device to access and get notifications from the remote server. The mobile computing device as herein referred to, includes but is not limited to, a smartphone, a laptop, a tablet, or similar client computing devices. To achieve this, the user may need to open a user account hosted on the remote server. This allows the user to receive personalized information about his or her pet.

Further, the user account may allow the user to receive notifications and monitor the plurality of biochemical sensors through a client device. On the device side, the controller processes the plurality of biochemical signatures to determine an illness diagnosis or send a warning notification to the user via the remote server.

Subsequently, the controller compares each biochemical signature with a corresponding biometric threshold range in order to identify at least one out-of-range biochemical signature from the plurality of biochemical signature (Step D). This allows the controller to determine if the animal is suffering from an illness. In one possible embodiment, the out-of-range biochemical signature may be high body temperature which would indicate a fever. This causes the controller to output a notification to the mobile computing device.

Accordingly, the controller generates a warning message if the out-of-range biochemical signature is identified during Step D (Step E). The warning message notifies the user if the arbitrary biochemical signature is dangerously high or low. The warning message may be embodied in an auditory alarm sounded by the mobile computing device. Further, the auditory alarm may be accompanied by a visual alarm displayed on the screen of the mobile computing device. In the preferred embodiment, the warning message may be sent as a short message service (SMS) to the user's smartphone. In an alternate embodiment, the warning message may be sent through a mobile application downloaded into the smartphone. This compels the user to tend to his or her pet.

Preferably, the remote server relays the warning message from the wireless communication module to the mobile computing device (Step F). More specifically, the wireless communication module sends the warning message to the remote server, which then transmits the warning message to the mobile computing device. In one possible embodiment, the controller also determines an illness diagnosis from the plurality of biochemical signatures. The illness diagnosis may be sent to the user with the warning message. In the preferred embodiment, the illness diagnosis is identified by the controller and transmitted to the remote server for distribution to the user. Alternately, the remote server may identify the illness diagnosis based on the plurality of biochemical signatures.

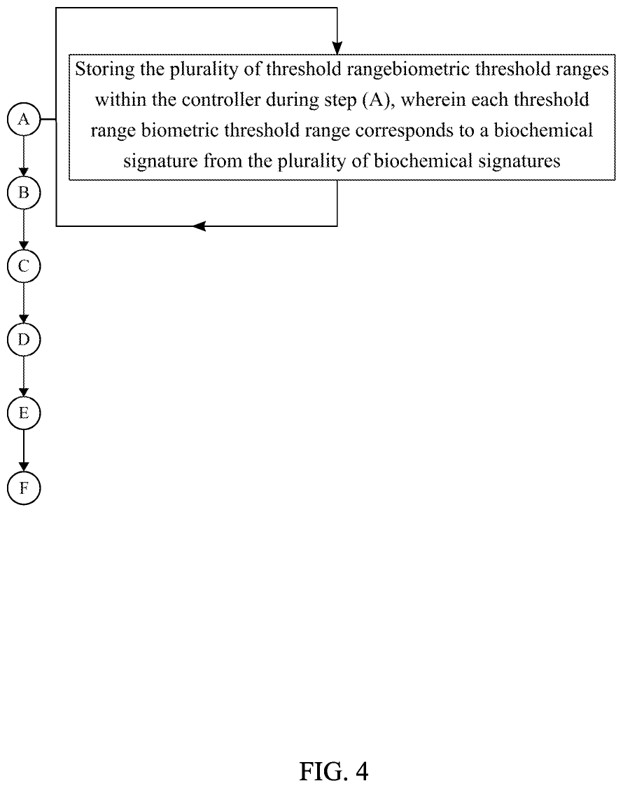

Referring to FIG. 4, in one embodiment, the plurality of biometric threshold ranges is stored within the controller during Step A, wherein each biometric threshold range corresponds to a biochemical signature from the plurality of biochemical signatures. For instance, the plurality of biometric threshold ranges may be stored in an onboard memory module of the controller. Further, in one embodiment, the memory may be overwritten to update the plurality of biometric threshold ranges. This may be achieved via over the air (OTA) updates sent from the remote server or manually through input/output (I/O) ports provided on the health monitoring device. The plurality of biometric threshold ranges may include a biometric threshold range for the heartbeat reading and the body temperature reading. This establishes the tolerable maximum and minimum for each biochemical signature.

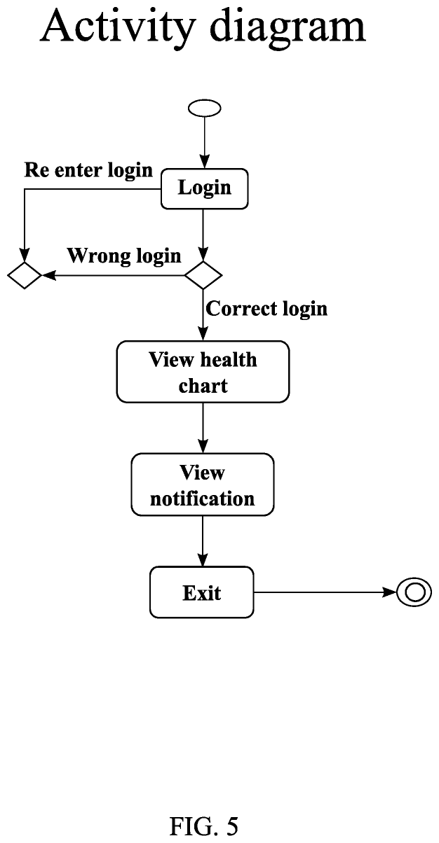

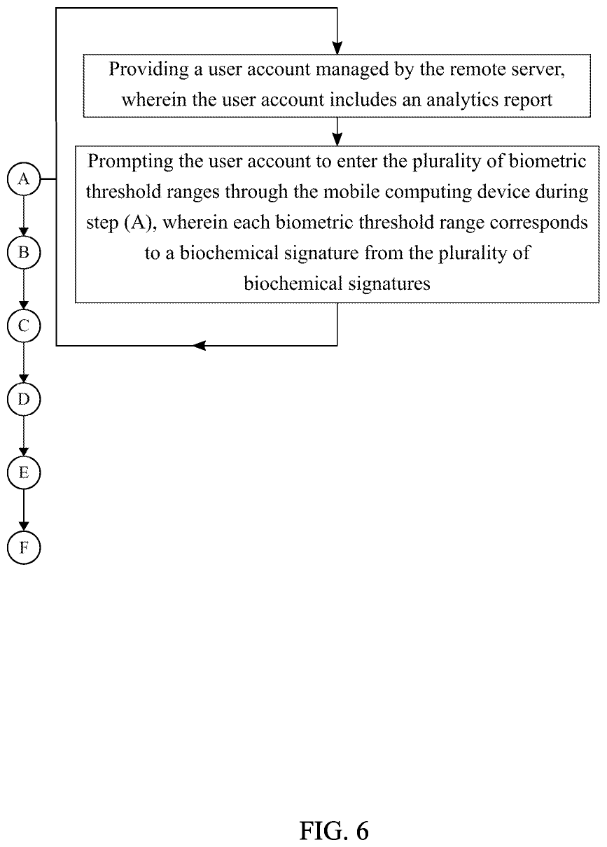

Referring to FIG. 5 and FIG. 6, in an alternate embodiment, the plurality of biometric threshold ranges is entered by the user. As such, the user account managed by the remote server is provided, wherein the user account includes an analytics report. The user account is registered by the user to access his or her pet's plurality of biochemical signatures stored in the remote server. Preferably, the user account is secured against unauthorized access by security credentials, such as a username, a password, security questions, and the like. In the preferred embodiment, the user account is provided as part of the mobile application downloaded in the user's mobile computing device. The mobile application allows the user to view a live feed of the plurality of biochemical signatures and view a past record of warning messages. In the preferred embodiment, the warning message is sent as a SMS message or a phone call to the mobile computing device. Alternately, the warning message may be sent as a visual or auditory alarm that plays through the mobile application. The analytics report contains insights into the overall health of the animal gained by studying the plurality of biochemical signatures over a prolonged period. Further, an admin account is provided to allow an administrator to manage the user accounts, applications, and databases in the remote server Like the user account, the admin account requires a username and a password to be opened. The admin account is preferably hosted on the remote server and may be accessed by an administrator via a client computing device such as a laptop.

Referring to FIG. 5, in one possible embodiment, the user must login to the mobile application via a username and password. If the username or password is not correct, the user is redirected to the login process. Once in the mobile application, the user can view the plurality of biochemical signatures in the form of a graph. Further, the user can also view a record of past warning messages sent by the wireless communication module.

Subsequently, the controller prompts the user account to enter the plurality of biometric threshold ranges through the mobile computing device during Step A, wherein each biometric threshold range corresponds to a biochemical signature from the plurality of biochemical signatures. More specifically, the plurality of biometric threshold ranges entered by the user account is relayed through the remote server to the wireless communication module. This allows the user to customize the plurality of biometric threshold ranges for his or her pet. As such, the user thus has greater control over what is considered to be safe threshold range.

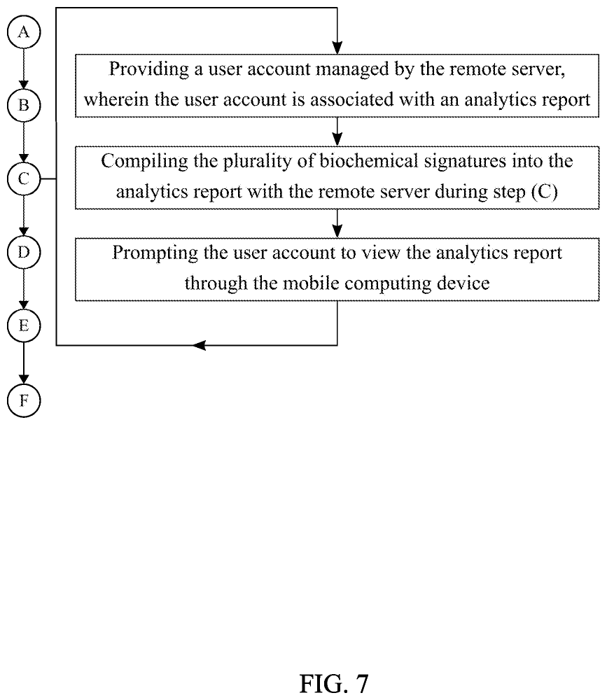

Referring to FIG. 7, further, the remote server compiles the plurality of biochemical signatures into the analytics report during step C. Subsequently, the mobile computing device prompts the user account to view the analytics report.

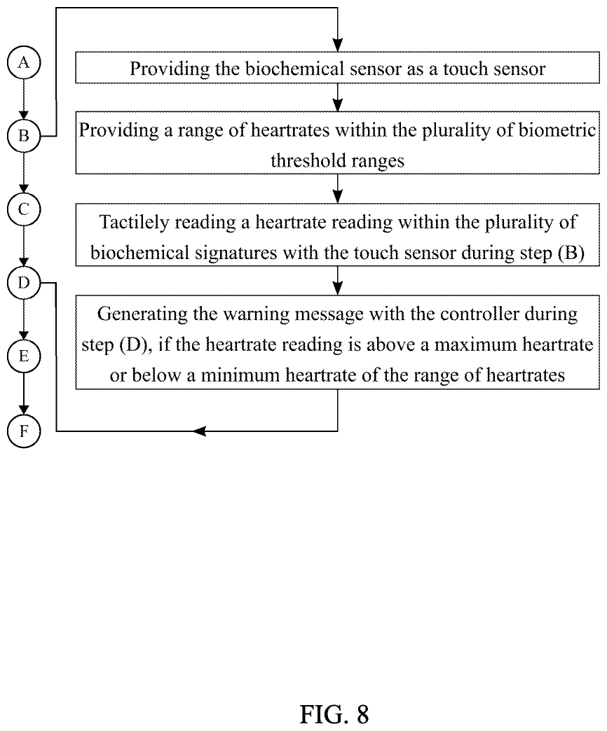

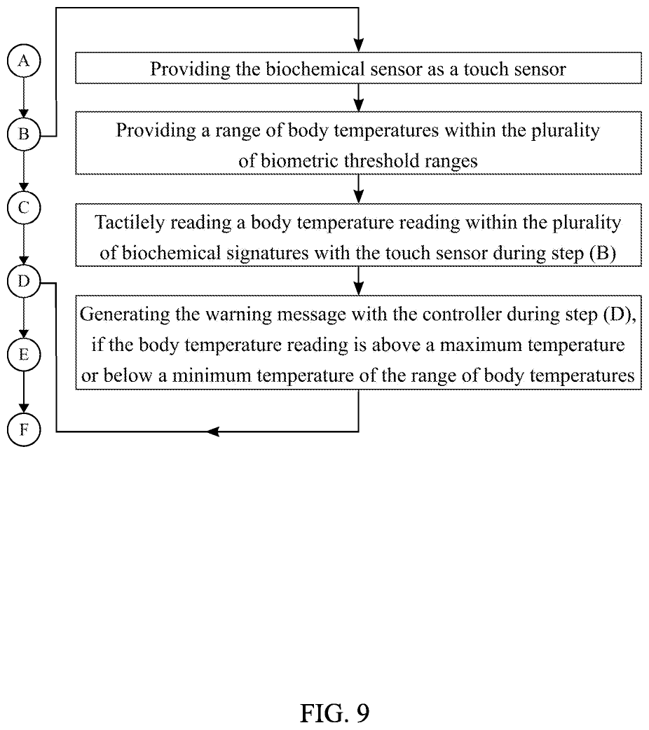

Referring to FIG. 8 and FIG. 9, in one possible embodiment of the present invention, a touch sensor is provided as the biochemical sensor. In this embodiment, a range of heartrates is provided within the plurality of biometric threshold ranges. Further, a range of body temperature is also provided within the plurality of biometric threshold ranges. As the name implies, the touch sensor contacts the skin of the animal. Accordingly, the touch sensor takes readings of the heart beat and the body temperature of the animal. As such, the touch sensor tactilely reads a heartrate reading within the plurality of biometric threshold signatures during Step B. Likewise, the touch sensor tactilely reads a body temperature reading within the plurality of biochemical signatures during Step B. In the preferred embodiment of the present invention, the touch sensor includes an optical hear-rate monitor which shines light into the animal's skin and process the reflected light to determine the heart rate. The touch sensor also includes a body temperature sensor. The preferred body temperature sensor may include a thermocouple and thermistor for generating accurate temperature readings. The thermistor is preferably positioned adjacent to the optical heart rate monitor, in contact with the animal's skin.

The heartrate reading is preferably taken by the optical heart-rate monitor and is measured in beats per minute (bpm). The body temperature reading refers to the internal body temperature of the animal. As such, the body temperature reading may be measured in degrees centigrade or Fahrenheit. Both the heartrate reading and the body temperature reading may be continuously or periodically transmitted to the remote server by the wireless communication module. Further, the heartrate reading and the body temperature reading are preferably archived on the remote server to provide the user with a historical database for reference.

In the preferred embodiment, the heartrate reading and the body temperature reading are displayed on separate graphs on the screen of the mobile computing device. The graph may display a scatter plot or a continuous line graph. The scatter plot may display the heartrate reading or the body temperature reading as data points taken over three-hour interval. For instance, the scatter plot may show the heartrate reading or the body temperature reading in the Y-axis and the time in the X-axis. Further, a line of best fit may be fitted on the data points. Alternately, each data point of the body temperature reading or the heartrate reading may be connected in a continuous line graph.

Preferably, the body temperature reading is taken by the thermistor and converted into a digital signal by the thermocouple. In this embodiment, the body temperature is taken on the neck of the animal. Alternately, the body temperature may be taken on the thigh, mouth, or any other body part of the animal. Accordingly, the plurality of biometric threshold ranges includes a range of heartrates and a range of body temperatures. Range of heartrates and the range of body temperatures establish the upper bound and the lower bound of safe body temperatures and heart rates.

In one possible embodiment, the range of heartrates and the range of body temperatures are stored in the onboard memory of the controller. In this embodiment, the range of heartrates and the range of body temperatures are determined by analyzing past medical records of analyzing past medical records of hundreds of animals of the same species, gender, and size as the pet animal.

In another possible embodiment, the range of heartrates and the range of body temperatures are entered by the user via the mobile computing device. In this embodiment, the user customizes the range of heartrates and the range of body temperatures for his or her pet. The user may consult an external source for information or may consult a veterinary doctor to determine the correct the range of heartrates and the range of body temperatures.

Accordingly, the controller generates the warning message during Step D, if heartrate reading is above a maximum heartrate or below a minimum heartrate of the range of heartrates. Similarly, the controller also generates the warning message during Step D, if the body temperature reading is above a maximum temperature or below a minimum temperature of the range of body temperatures. The maximum heartrate refers to the upper bound of the range of heart rates. In contrast, the minimum heartrate refers to the lower bound of the range of heart rates. Similarly, the maximum heartrate refers to the upper bound of the range of body temperatures. In contrast, the minimum heartrate refers to the lower bound of the range of body temperatures.

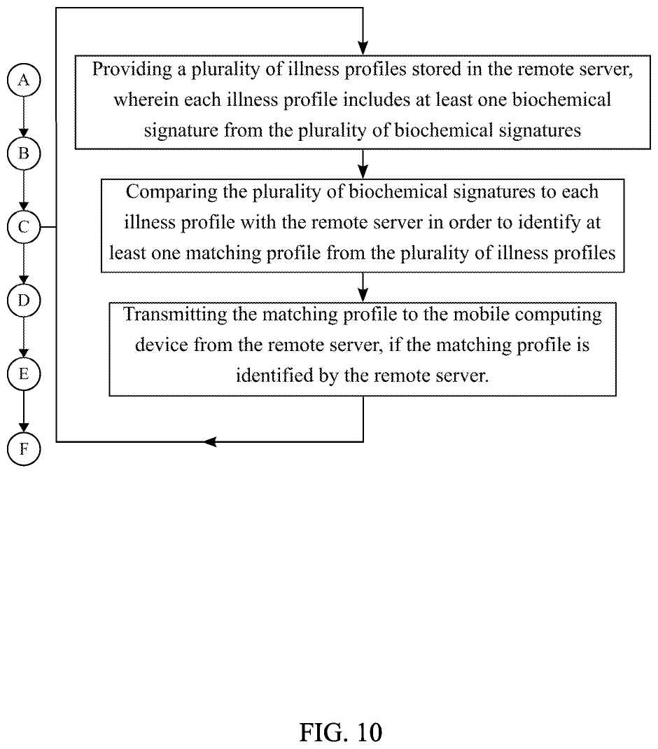

Referring to FIG. 10, in a possible embodiment, the controller provides an illness diagnoses based on the plurality of biochemical signatures. Accordingly, a plurality of illness profiles stored in the remote server is provided, wherein each illness profile includes at least one biochemical signature from the plurality of biochemical signatures. Alternately, the illness profile may be associated with more than one biochemical signature. Each illness profile is associated with a biochemical signature based on historical medical records and research. For instance, an illness profile for canine influenza may include an increased body temperature. The biochemical signature for each illness profile is validated against prior medical records for animals of the same species, gender, body weight, and age as the pet. Accordingly, the remote server compares the plurality of biochemical signatures to each illness profile from the plurality of illness profiles. More specifically, the remote server compares the plurality of biochemical signatures to the corresponding biochemical signature included in each illness profile, in order to identify at least one matching profile from the plurality of illness profiles. More specifically, the matching profile contains one or more biochemical signature that is numerically equivalent to the one or more biochemical signature taken by the biochemical sensor. Finally, the remote server transmits the matching profile to the mobile computing device, if the matching profile is identified by the remote server.

The preferred embodiment of the present invention utilizes a pet-collar for a pet like a dog or a cat. The pet-collar is a plastic or fabric strap that is tied around the neck of the animal. Preferably, the pet-collar is secured with a buckle or a snap that prevents the animal from releasing the pet collar. In the preferred implementation, the pet-collar securely attaches the biochemical sensor to the animal. More specifically, the health monitoring device is mounted to the pet-collar with the biochemical sensor facing inwards. This allows the biochemical sensor to contact the skin of the animal and take the necessary readings. In alternate embodiments of the present invention, the health monitoring device may be mounted onto a leg brace or a patch which can be mounted anywhere on the animal's body. This allows the biochemical sensor to obtain high-quality readings of the plurality of biochemical signatures.

In another embodiment of the present invention, the health monitoring device may comprise a Global Positioning System (GPS) transceiver. The GPS transceiver transmits the GPS coordinates of the animal to the remote server via the wireless communication module. The GPS coordinates are then stored in the database of the remote server and sent to the mobile computing device. In the preferred embodiment, the GPS coordinates of the animal may be displayed on a map on the screen of the mobile communication device. This allows the user to track the location of the animal and prevent the animal from straying too far from the owner.

Although the invention has been explained in relation to its preferred embodiment, it is to be understood that many other possible modifications and variations can be made without departing from the spirit and scope of the invention as hereinafter claimed.

What is claimed is:

1. A method for pet-health awareness with real-time health monitoring and illness notification functions comprises:
(A) providing a heath monitoring device, at least one remote server, and a mobile computing device, wherein the heath monitoring device includes a controller, a wireless communication module, at least one biochemical sensor, and wherein the controller stores a plurality of biometric threshold ranges;
(B) reading a plurality of biochemical signatures with the biochemical sensor;
(C) relaying the plurality of biochemical signatures from the biochemical sensor to the remote server through the wireless communication module;
(D) comparing each biochemical signature with a corresponding biometric threshold range with the controller in order to identify at least one out-of-range biochemical signature from the plurality of biochemical signature;
(E) generating a warning message with the controller, if the out-of-range biochemical signature is identified during step (D); and
(F) relaying the warning message from the wireless communication module to the mobile computing device through the remote server.

2. The method for pet-health awareness with real-time health monitoring and illness notification functions as claimed in claim 1 comprises:
storing the plurality of biometric threshold ranges within the controller during step (A), wherein each biometric threshold range corresponds to a biochemical signature from the plurality of biochemical signatures;

3. The method for pet-health awareness with real--time health monitoring and illness notification functions as claimed in claim 1 comprises:
providing a user account managed by the remote server, wherein the user account includes an analytics report; and
prompting the user account to enter the plurality of biometric threshold ranges through the mobile computing device during step (A), wherein each biometric threshold range corresponds to a biochemical signature from the plurality of biochemical signatures.

4. The method for pet-health awareness with real-time health monitoring and illness notification functions as claimed in claim 1 comprises:
providing a user account managed by the remote server, wherein the user account is associated with an analytics report;
compiling the plurality of biochemical signatures into the analytics report with the remote server during step (C); and
prompting the user account to view the analytics report through the mobile computing device.

5. The method for pet-health awareness with real-time health monitoring and illness notification functions as claimed in claim 1 comprises:
providing the biochemical sensor as a touch sensor;
providing a range of heartrates within the plurality of biometric threshold ranges;
tactilely reading a heartrate reading within the plurality of biochemical signatures with the touch sensor during step (B); and
generating the warning message with the controller during step (D), if the heartrate reading is above a maximum heartrate or below a minimum heartrate of the range of heartrates.

6. The method for pet-health awareness with real-time health monitoring and illness notification functions as claimed in claim 1 comprises:
providing the biochemical sensor as a touch sensor;
providing a range of body temperatures within the plurality of biometric threshold ranges;
tactilely reading a body temperature reading within the plurality of biochemical signatures with the touch sensor during step (B); and
generating the warning message with the controller during step (D), if the body temperature reading is above a maximum temperature or below a minimum temperature of the range of body temperatures.

7. The method for pet-health awareness with real-time health monitoring and illness notification functions as claimed in claim 1 comprises:
providing a plurality of illness profiles stored in the remote server, wherein each illness profile includes at least one biochemical signature from the plurality of biochemical signatures;
comparing the plurality of biochemical signatures to each illness profile with the remote server in order to identify at least one matching profile from the plurality of illness profiles; and
transmitting the matching profile to the mobile computing device from the remote server, if the matching profile is identified by the remote server.
